(19) United States
(12) Patent Application Publication
(10) Pub. No.: US 2016/0213863 A1
(43) Pub. Date: Jul. 28, 2016

(54) Droplet Medicament Delivery Device

(71) Applicant: CAREBAY EUROPE LTD, Swatar (MT)

(72) Inventors: Marcus Söderlund, Sköndal (SE); Wilhelmus P.J. de Kruif, Enschede (NL)

(73) Assignee: Carebay Europe Ltd, Sliema (MT)

(21) Appl. No.: 14/915,241

(22) PCT Filed: Aug. 21, 2014
(86) PCT No.: PCT/EP2014/067829

§ 371 Date: Feb. 28, 2016

(30) Foreign Application Priority Data
Aug. 29, 2013 (SE) 1350997-1

(51) Int. Cl. A61M 11/00; A61M 39/24; G01F 11/02

(52) U.S. Cl. CPC A61M 11/007; G01F 11/025; A61M 39/24; A61M 2205/43

(57) ABSTRACT

A droplet medicament delivery device for delivering a volume of a medicament from a reservoir. The device includes a device body; a valve chamber surrounded by a chamber wall movably arranged in the device body between at least a first position and a second position; at least one passage extending through the chamber wall; a valve; a valve outlet; a droplet generating device connected to the valve outlet; and an actuator arranged in the valve chamber. The actuator, when activated, delivers medicament from the valve chamber via the valve outlet. The valve inlet is open to the valve chamber and the valve outlet is closed when the chamber wall is in the first position, and the valve inlet is closed and the valve outlet is open when the chamber wall is in the second position.

### FIELD OF THE INVENTION

The present invention relates to a droplet medicament delivery device for delivery of a volume of medicament from a reservoir.

### BACKGROUND OF THE INVENTION

Different types of medicaments are frequently delivered to persons suffering from different diseases to cure, or at least to reduce, their symptoms. In order to deliver multiple doses of medicament, a predetermined volume of the medicament is extracted from a reservoir and upon activation of the delivery equipment delivered to the intended person.

There are a number of different devices such as reservoirs, containers, valves and different types of dose delivery devices intended to facilitate the administration of regularly delivered doses of medicament.

Examples of already known devices for dose delivery are disclosed for example in U.S. Pat. No. 7,387,121, which discloses a delivery device that extracts a metered volume of medicament, intended to be delivered to a user. The disclosed device, however, has a complex design and there is a risk that the metered doses do not always have the same volume due to a tolerance chain of many parts and air entrapment in the device.

Another example of a delivery device is disclosed in WO2010/077278. The disclosed device however has a complex design and the large air entrapment will have effect on dose accuracy.

Consequently, known devices suffer from some severe drawbacks. First, all of them have a complex design that makes them expensive to manufacture, and in some cases also difficult for the user to operate. Secondly, the complex designs, in combination with a reservoir large enough to store multiple doses, result in large dose delivery devices which makes them uncomfortable and difficult for the user to carry in, for instance, a pocket etc.

There is consequently a need for improvement of the droplet delivery device in order to reduce the drawbacks described above.

### SUMMARY OF THE INVENTION

The present invention, defined in the appended claims, provides a liquid droplet medicament delivery device for delivery of a volume of a medicament from a reservoir that to at least some extent fulfils the needs defined above.

The liquid droplet medicament delivery device comprises:
a device body;
a valve chamber arranged in said device body, said valve chamber is surrounded by a chamber wall movably arranged in the device body between at least a first and a second position;
a passage extending through the chamber wall;
a valve inlet extending in the device body from the reservoir to the chamber wall;
a valve outlet extending in the device body from the chamber wall;
a droplet generating device connected to the valve outlet; and
an actuator arranged in the valve chamber, said actuator, when activated, delivers medicament from the valve chamber via the valve outlet,
wherein the valve inlet is open to the valve chamber and the valve outlet is closed when the chamber wall is in the first position, and the valve inlet is closed and the valve outlet is open when the chamber wall is in the second position.

The liquid droplet medicament delivery device is prepared for use by arranging the chamber wall in the first position to open the valve inlet and fill the valve chamber by medicament from the storage reservoir. When the desired volume has entered the valve chamber, the chamber wall is moved to the second position in order to close the valve inlet and open the valve outlet. The medicament enclosed in the valve chamber is thereby prepared for delivery by the actuator that upon activation forces the medicament out of the valve chamber via the valve outlet and the droplet generating device, to the user.

The claimed liquid droplet medicament delivery device fulfils the needs defined above by providing a liquid droplet medicament delivery device design with reduced complexity that still ensures a reliable and precise delivery of a predetermined volume of medicament from a larger storage reservoir. The valve could be used a number of times and still ensure the desired preciseness of the delivered volume.

The use of a liquid droplet medicament delivery device comprising a valve chamber for the desired volume of medicament makes it possible to reduce the complexity of the design considerably since the force required to achieve the desired dispensing pressure on the medicament in the valve chamber is considerably smaller than what would have been required if the dispensing were to take place directly from the larger storage reservoir at the same dispensing pressure.

A further advantage with the claimed device is that the valve chamber may be arranged separate from the storage reservoir which could be beneficial if the valve chamber and the storage reservoir are fitted in for example a portable handheld device where the available space is limited.

In one embodiment of the delivery device, the valve chamber has a longitudinal axis (A) and a substantially circular cross section transverse to the longitudinal axis (A), said chamber wall is rotatable about the longitudinal axis (A) between the first and second positions. This embodiment of the device provides the desired functionality with a limited amount of different components.

In one embodiment of the delivery device, the actuator comprises a substantially circular plunger with a diameter corresponding to the diameter of the valve chamber, said plunger is movable back-and-forth in the valve chamber, co-axially with the longitudinal axis (A). This embodiment provides an actuator that is able to expel the medicament in the valve chamber in a reliable way under the desired pressure required to ensure that droplets are generated and delivered.

In one embodiment of the delivery device, the plunger is movable between a depressed position, in which the valve chamber is emptied, and a retracted position, in which the valve chamber has a predetermined volume. The two defined positions ensure that the desired predetermined volume of medicament could be filled and metered in the valve chamber before it is delivered under pressure.

In one embodiment of the delivery device, the actuator furthermore comprises a plunger movement mechanism that upon activation moves the plunger to the depressed position. The movement mechanism ensures that the medicament is delivered with the desired pressure each time the delivery device is used.

In one embodiment of the delivery device, the plunger movement mechanism furthermore is able to, upon activation, move the plunger from the depressed position to the retracted position. This embodiment of the delivery device reduces the time required to fill the valve chamber by medicament since the filling of the chamber with medicament from the reservoir is assisted by the movement mechanism.

In one embodiment of the delivery device, a sound generator is connected to the actuator to generate a sound when the plunger reaches the retracted position in order to inform the user that the delivery device is prepared for activation.

In one embodiment of the delivery device, the chamber wall furthermore is movable, e.g. turnable, to a third position in which none of the valve inlet or valve outlet is open. This embodiment is favourable since it reduces the time required to prepare the device for use. This is achieved since the valve chamber could be filled with medicament and the chamber wall turned to the third position in which both the valve inlet and outlet are closed until the user needs to use the device.

In one embodiment of the delivery device, the droplet generating device is a perforated chip or nozzle that generates the droplets. Both these alternatives effectively generate droplets with the desired size at the delivery pressure obtainable by the present actuator.

One embodiment of the delivery device furthermore comprises the reservoir containing the medicament as an integrated part of the device. This embodiment is advantageous if the delivery device is disposable, e.g. if it is only intended to be used for delivery of a predetermined number of doses corresponding to the volume of the reservoir.

In one embodiment of the delivery device, the delivery device comprises means for continuously applying a pressure on a medicament when present in the reservoir. This embodiment is favourable in that the applied pressure ensures that the filling of the valve chamber is initiated immediately when the chamber wall is arranged in the first position which reduces the time required to prepare the device for use.

One embodiment of the delivery device furthermore comprises an outer casing enclosing the reservoir and the device body. This embodiment is favourable since this makes it possible for the user to carry the delivery device in, for example, a pocket or a bag without risk of damaging the different components of the device. The casing could also be designed with an attractive and comfortable shape that facilitates the use of the delivery device.

One embodiment of the delivery device furthermore comprises an actuator activation device accessible from the outside of the casing to activate the dispensing of medicament droplets from the device in order to facilitate the use of the delivery device.

The different embodiments described above could of course be combined and modified in different ways without departing from the scope of the invention that will be described in more detail in the detailed description.

### BRIEF DESCRIPTION OF THE DRAWINGS

Different embodiments of a delivery device according to the present invention are illustrated in the appended figures.

FIG. 1 schematically illustrates a cross-sectional view of a first embodiment of a delivery device.

FIGS. 2a to 2f schematically illustrates the delivery device during use.

### DETAILED DESCRIPTION

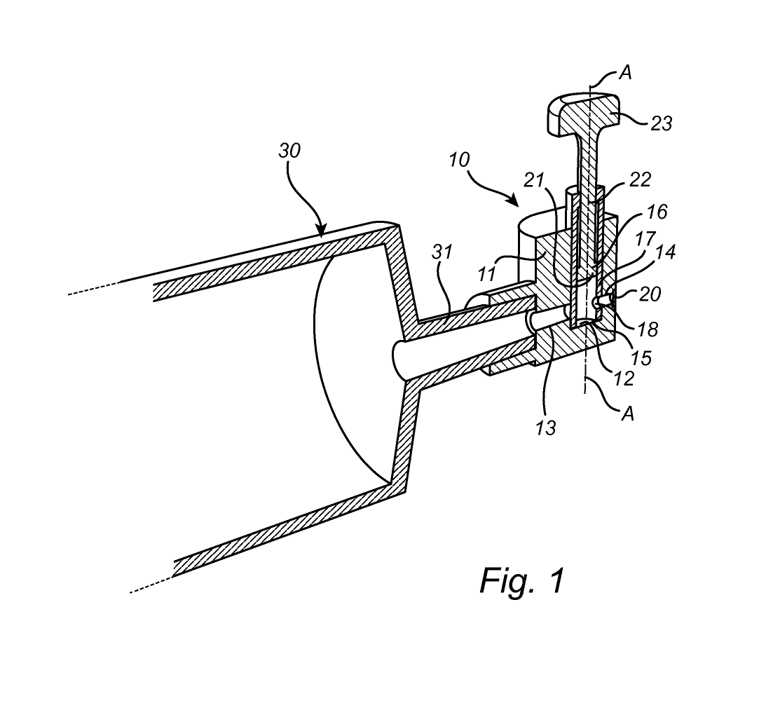

In FIG. 1, selected components of a first embodiment of a delivery device 10 according to the invention are illustrated schematically. The delivery device 10 is arranged to receive medicament from a reservoir 30. The reservoir is large enough to contain a number of doses that upon activation of the delivery device is dispensed to a user. The reservoir 30 and the delivery device 10 are preferably surrounded by a protective outer casing enclosing the reservoir and the device body to protect the different components from damage.

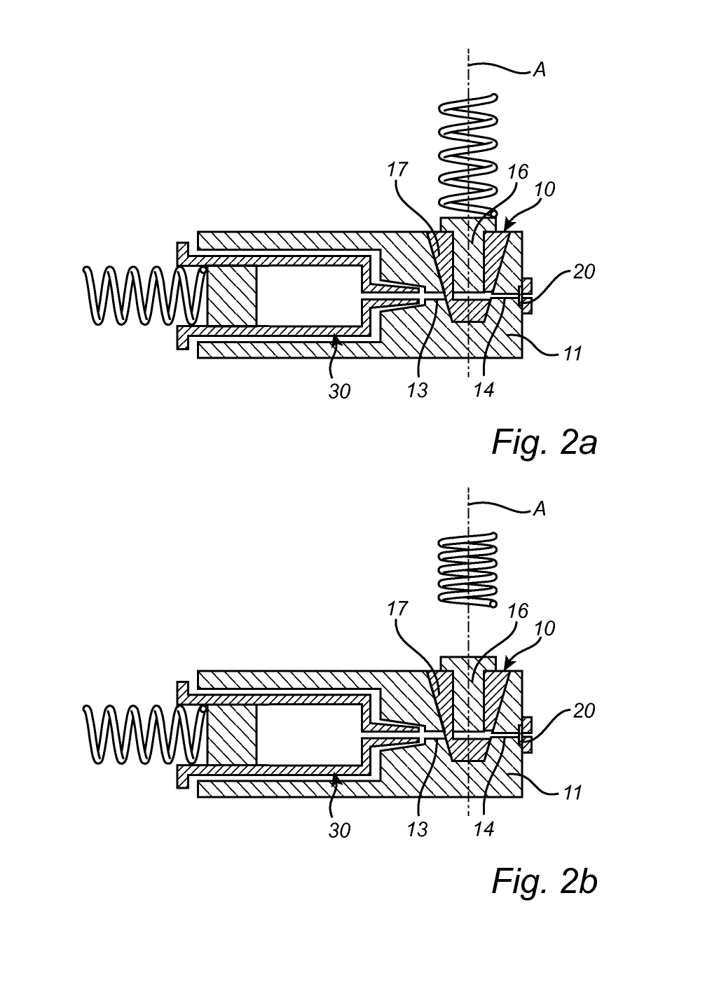

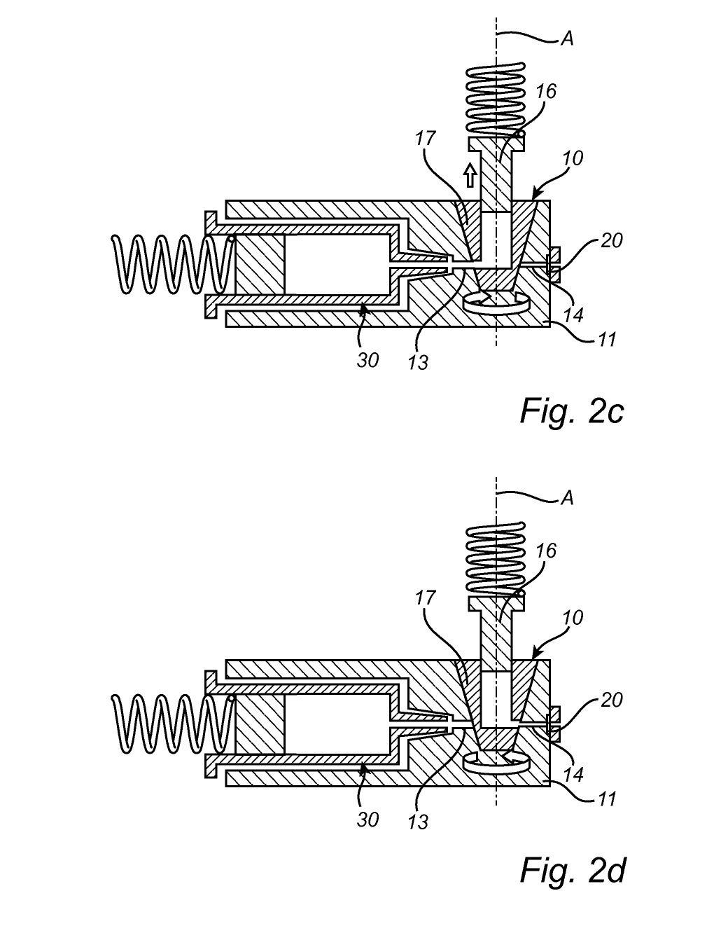

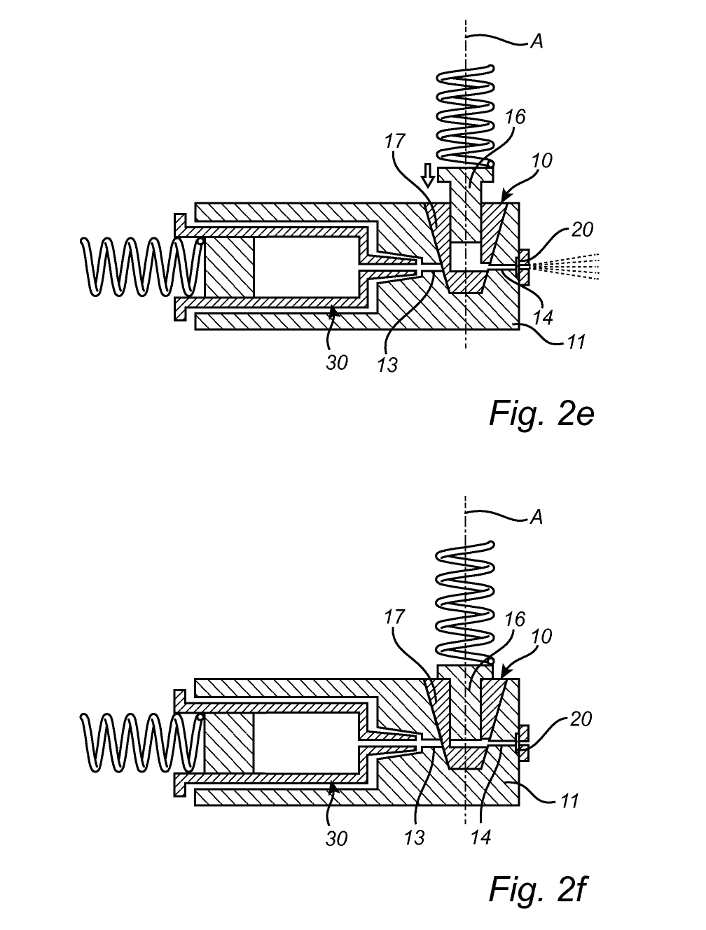

The reservoir 30 has a dispensing end formed as a pipe 31 connected to the delivery device 10 in order to deliver medicament to the delivery device. Preferably a reservoir plunger, piston and a spring act on the medicament in the reservoir to continuously apply a pressure on the medicament and facilitate the delivery of medicament from the reservoir to the delivery device. This is schematically illustrated in FIG. 2. However, the size and shape of the reservoir could be adapted to different types of medicaments and needs.

The delivery device comprises a device body 11 and a valve chamber 12 formed within the device body 11. The valve chamber 12 has a longitudinal axis A and a substantially circular cross section transverse to the longitudinal axis A. The diameter of the valve chamber as well as the length of the valve chamber along the longitudinal axis A is selected to provide the desired chamber volume which in the end is equal to the volume of each dose delivered from the delivery device when the device is activated. The illustrated valve chamber has a circular cross-section but other shapes like oval, conical etc are also possible as long as the function of the valve is ensured. The valve chamber 12 has a lower stationary circular end surface 15 which is formed in the device body 11. The upper end of the valve chamber 12 is closed by an actuator 16 movably arranged along the longitudinal axis A within the valve chamber 12 for dispensing of medicament from the valve chamber 12. The actuator 16 will be described in more detail below.

The valve chamber is surrounded by a chamber wall 17 extending from the lower stationary end surface 15 and well past the upper position of the actuator 16. The chamber wall 17 is in the illustrated embodiment substantially tube-shaped and rotatably arranged about the longitudinal axis A. A passage 18 extends through the chamber wall 17 close to the stationary end surface. The size and position of the passage 18 along the longitudinal axis A corresponds to the position and size of a valve inlet 13 extending within the device body 11 from the reservoir 30 to the chamber wall, as well as a valve outlet 14 also arranged in the device body 11 and extending from the chamber wall 17 to an exterior of the device, via a droplet generating device connected to the outlet, to be explained below.

The valve inlet 13 and valve outlet 14 are arranged in the same plane transverse to the longitudinal axis A, and the chamber wall 17 is arranged rotatable about the longitudinal axis A between a first position in which the passage 18 is aligned with the valve inlet 13, and a second position in which the passage 18 is aligned with the valve outlet 14. The chamber wall 17 may also be positioned in a third position in which the passage 18 not is aligned with anyone of the valve inlet or outlet to provide a closed valve chamber.

In one preferred embodiment of the device, the chamber wall is furthermore turnable to a third position in which none of the valve inlet or valve outlet is open. This third position makes it possible to close both the valve inlet and outlet and prevent leakage and drooling from the delivery device. The turning of the chamber wall could be controlled by a valve control device arranged to control the movement of the chamber wall between the different positions of the chamber wall.

The schematically illustrated actuator 16 comprises a plunger 21 with a shape and size corresponding to the cross-sectional shape of the valve chamber to provide a movable seal at one end of the valve chamber. From the plunger 21 a plunger rod 22 extends coaxially with the longitudinal axis A of the valve chamber to a position where it is reachable by the user if the plunger rod is ended by a button 23 for actuation of the dose delivery. Alternatively, the plunger rod is ended by a powerpack controlled by an actuation lever located somewhere on the device where it is accessible for the user. The plunger 21 is movable between a depressed position DP in which the valve chamber is emptied, i.e. has a very small volume, and a retracted position RP in which the valve chamber has a predetermined volume equal to the desired volume of a dose of medicament stored in the reservoir. The user initiates the dispensing of medicament from the delivery device by pressing the button 23 and thereby applying a pressure on the medicament in the valve chamber 12 to dispense the medicament via the valve outlet 14.

In an alternative embodiment of the valve chamber the size of the chamber may be set to a predetermined volume by controlling the range of movement of the actuator along the axial direction of the valve chamber. In this embodiment, the retracted position of the plunger may be set to a predetermined position to correspond to a predetermined dose. In one embodiment an axially movable blocking element (not shown), having visual indicia, is arranged to limit the movement of the plunger and to set the retracted position RP of the plunger to predetermined amount.

The delivery device 10 furthermore comprises the droplet generating device 20, mentioned above, connected to the valve outlet 14. The droplet generating device is either a perforated chip or a nozzle that, when the medicament is supplied under pressure, generates droplets and dispenses them to a user in need of a dose of medicament. The chip or nozzle is adapted to the specific conditions of the medicament to form droplets of desired size. The droplet generating device may, for instance, be a semiconductor chip that comprises perforations, such as micro channels, having a predetermined configuration to form droplets of the desired size.

FIGS. 2a to 2f disclose, schematically, the different operational steps that take place during use of the delivery device 10. The delivery device in FIG. 2 is a slightly modified embodiment of the delivery device illustrated in FIG. 1 but works in a corresponding way.

In FIG. 2a, the delivery device according to the invention is illustrated after it has been used, and a dose has been delivered. The chamber wall 17 is located in the second position P2, i.e. the outlet 14 is open, and the plunger is located in the depressed position DP, which indicates that a dose of medicament has been delivered.

When there is a need for a dose from the delivery device 10 the chamber wall 17 is turned to the first position P1 in order to align the passage 18 in the chamber wall 17 with the valve inlet 13 and connect the delivery device 10 with the reservoir 30, as illustrated in FIG. 2c. When the chamber wall is turned to the first position, the valve outlet 14 is closed by the chamber wall 17. As soon as the valve inlet 13 and the passage 18 are aligned, the filling of the valve chamber 12 with medicament from the reservoir 30 is initiated automatically if the medicament in the reservoir is pressurized, alternatively manually by the operator that activates the filling. When the entire valve chamber 12 is filled with medicament, i.e. the plunger has moved all the way to the retracted position RP, the chamber wall 17 is turned to the second position P2, i.e. the passage 18 in the chamber wall 17 is aligned with the valve outlet 14, as illustrated in FIG. 2d, and the chamber valve closes the valve inlet simultaneously to prevent a backflow of medicament from the chamber to the reservoir during delivery of the dose. The delivery device 10 is now prepared for use.

In order to activate the delivery of a dose of medicament from the delivery device 10, the user activates the actuator in the valve chamber 12 and the plunger starts to move along the longitudinal axis A. This means that a pressure is applied to the medicament in the chamber 12 and the medicament is dispensed via the valve outlet 14 to the droplet generating device connected to the valve outlet, as illustrated in FIG. 2e.

When the plunger reaches the depressed position DP, the entire dose of medicament has been delivered, as illustrated in FIG. 2f.

Preferably, the chamber wall is movable to a third position P3 as well. In such a case, the chamber wall is turned to the third position after the delivery has been completed to close the valve outlet and prevent drooling from the delivery device until additional doses are needed.

Finally, it is realized that the illustrated embodiments of the actuator are very simple and more complex solutions are also possible, and in some cases preferred. For example, the actuator could comprise a plunger movement mechanism, such as a spring-driven powerpack, that upon activation automatically moves the plunger to the depressed position and expels the medicament from the valve chamber under the desired pressure. Preferably the actuator is also able to, upon activation, move the plunger from the depressed position to the retracted position in order to assist the filling of the valve chamber with medicament from the reservoir 30. These preferred embodiments of the actuator could be achieved by incorporating one or more springs that upon activation applies a force to the plunger in the desired direction.

Preferred embodiments of the actuator furthermore comprise one, or more, sound generator connected to the actuator in order to communicate the actual position of the actuator, or that the filling of the chamber and/or the delivery of a dose of medicament has been completed as well as visual indicators to display the status of the device. The sound generator could be embodied in several ways, for example as a flexible part that stores energy during movement of the plunger and releases the stored energy when the plunger reaches the end position and the flexible part is released. This solution is sometimes called a click-finger, and a visual indicator may, for instance, be a window through which different colours can be displayed, depending on the position of the actuator. The delivery device could furthermore comprise an actuator activation device, such as a button, accessible from the outside of the surrounding casing where it is easy for the user to reach and thereby facilitate the activation of the delivery device and the dispensing of the doses of medicament. The activation may thus be either manual, such as pushing the button and consequently also the actuator, or automatic, such as releasing a tensioned spring for pushing the actuator.

Even though certain embodiments of the delivery device have been described and illustrated several modifications are possible. For example, the chamber wall could comprises two passages extending through the chamber wall at different axial positions, and the valve inlet and outlet arranged at different axial positions corresponding to the positions of the passages in the chamber wall to be opened and closed by turning the chamber wall between the first and second position. Thereby, filling of the chamber could be carried out during rotation of the valve.

The different embodiments described above could all be combined and modified in different ways without departing from the scope of the invention that is defined by the appended claims.

1.-15. (canceled)

16. A droplet medicament delivery device for delivering a volume of a medicament from a reservoir, the device comprising:
a body;
a valve chamber arranged in the body and surrounded by a chamber wall movably arranged in the body between at least a first position and a second position;
at least one passage extending through the chamber wall;
a valve inlet extending in the body from the reservoir to the chamber wall;
a valve outlet extending in the body from the chamber wall;
a droplet generator connected to the valve outlet; and
an actuator arranged in the valve chamber, wherein the actuator, when actuated, delivers medicament from the valve chamber via the valve outlet;
wherein the valve inlet is open to the valve chamber and the valve outlet is closed when the chamber wall is in the first position, and the valve inlet is closed and the valve outlet is open when the chamber wall is in the second position.

17. The delivery device of claim 16, wherein the at least one passage is aligned with the valve inlet when the chamber wall is in the first position, and the at least one passage is aligned with the valve outlet when the chamber wall is in the second position.

18. The delivery device of claim 16, wherein the valve chamber has a longitudinal axis and a substantially circular cross section transverse to the longitudinal axis, and the chamber wall rotates around the longitudinal axis between the first position and the second position.

19. The delivery device of claim 18, wherein the actuator comprises a substantially circular plunger with a diameter corresponding to a diameter of the valve chamber, and the plunger is coaxially movable in the valve chamber along the longitudinal axis.

20. The delivery device of claim 19, wherein the plunger is movable between a depressed position, in which the valve chamber is empty, and a retracted position, in which the valve chamber has a predetermined volume.

21. The delivery device of claim 20, wherein the actuator further comprises a plunger movement mechanism that upon activation moves the plunger to the depressed position.

22. The delivery device of claim 21, wherein the plunger movement mechanism, upon activation, moves the plunger from the depressed position to the retracted position.

23. The delivery device of claim 22, wherein a sound generator is connected to the actuator and generates a sound when the plunger reaches the retracted position.

24. The delivery device of claim 18, wherein the chamber wall is turnable to a third position, in which both the valve inlet and the valve outlet are closed.

25. The delivery device of claim 24, further comprising a valve control configured to control movement of the chamber wall between the positions of the chamber wall.

26. The delivery device of claim 16, wherein the droplet generator comprises a perforated chip or a nozzle.

27. The delivery device of claim 16, further comprising the reservoir.

28. The delivery device of claim 27, further comprising a mechanism configured to continuously apply a pressure on a medicament when present in the reservoir.

29. The delivery device of claim 27, further comprising an outer casing enclosing the reservoir and the body.

30. The delivery device of claim 27, further comprising an actuator activation device accessible from outside the outer casing to activate delivery of medicament droplets from the droplet medicament delivery device.
